(19) United States
(12) Patent Application Publication
(10) Pub. No.: US 2024/0316367 A1
(43) Pub. Date: Sep. 26, 2024

(54) HISTOTRIPSY EXCITATION SEQUENCES OPTIMIZED FOR BUBBLE CLOUD FORMATION USING SHOCK SCATTERING

(71) Applicants: HISTOSONICS, INC., Plymouth, MN (US); THE REGENTS OF THE UNIVERSITY OF MICHIGAN, Ann Arbor, MI (US)

(72) Inventors: Jonathan M. CANNATA, Ann Arbor, MI (US); Timothy L. HALL, Ann Arbor, MI (US); Adam D. MAXWELL, Ann Arbor, MI (US); Dejan TEOFILOVIC, Ann Arbor, MI (US)

(21) Appl. No.: 18/737,746

(22) Filed: Jun. 7, 2024

Related U.S. Application Data
(63) Continuation of application No. 16/410,821, filed on May 13, 2019.
Application No. 16/410,821 is a continuation of application No. 14/323,693, filed on Jul. 3, 2014, now Pat. No. 10,293,187.
(60) Provisional application No. 61/842,820, filed on Jul. 3, 2013.

(51) Int. Cl. A61N 7/00; A61B 17/00; A61B 17/22; A61B 17/225

(52) U.S. Cl. CPC A61N 7/00; A61B 17/22004; A61B 2017/00176; A61B 2017/22008; A61B 2017/22028; A61B 17/225; A61N 2007/0039; A61N 2007/0078

(57) ABSTRACT

Methods and devices for producing cavitation in tissue are provided. In one embodiment, a shock scattering method of Histotripsy therapy comprises delivering an initiation pressure waveform from an ultrasound therapy transducer into tissue, the initiation pressure waveform being configured to produce at least one bubble in the tissue, delivering a scattering pressure waveform from the ultrasound therapy transducer into the at least one bubble within a life-cycle of the at least one bubble, and producing cavitation nuclei near the at least one bubble with the scattering pressure waveform. The scattering pressure waveform can be delivered during the life-cycle of the at least one bubble. In some embodiments, the scattering pressure waveform is delivered within 5 μs to 1 s of the initiation pressure waveform. Systems for performing shock scattering Histotripsy therapy are also discussed.

### CROSS REFERENCE TO RELATED APPLICATIONS

This application is a continuation of U.S. patent application Ser. No. 16/410,821, filed May 12, 2019, which is a continuation of U.S. patent application Ser. No. 14/323,693, filed Jul. 3, 2014, now U.S. Pat. No. 10,293,187; which application claims the benefit under 35 U.S.C. 119 of U.S. Provisional Patent Application No. 61/842,820, filed Jul. 3, 2013, titled “Modulated Excitation Sequences for Enhanced Pulsed Ultrasound Cavitational Therapy”, which applications are incorporated herein by reference.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure generally relates to treating tissue with cavitation created by ultrasound therapy.

### BACKGROUND

Histotripsy, or pulsed ultrasound cavitation therapy, is a technology where short, intense bursts of acoustic energy induce controlled cavitation (microbubble or bubble cloud formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high pressure amplitude acoustic pulses with low duty cycle.

Compared with conventional focused ultrasound technologies, Histotripsy has important advantages: 1) the destructive process at the focus is mechanical, not thermal; 2) bubble clouds appear bright on ultrasound imaging thereby confirming correct targeting and localization of treatment; 3) treated tissue appears darker (hypoechoic) on ultrasound imaging, so that the operator knows what has been treated; and 4) Histotripsy produces lesions in a controlled and precise manner. It is important to emphasize that unlike microwave, radiofrequency, or high-intensity focused ultrasound (HIFU), Histotripsy is not a thermal modality.

Early canine studies of Histotripsy homogenization of prostate tissue employed a therapy transducer that was positioned to deliver Histotripsy transabdominally. In these studies, the prostate was located only a short distance from the skin surface and there was a relatively wide path from the transducer through the skin to focus ultrasound energy. Consequently, the spherical Histotripsy therapy transducer employed in these studies had 14 cm aperture and 10 cm focal length (F-number=0.71). Histotripsy therapy transducers with high F-numbers have very low efficiency compared to transducers with low F-numbers. These inefficiencies are primarily due to nonlinear acoustic propagation leading to shockwave formation.

Specialized therapy transducer and drive electronics have been designed to focus Histotripsy therapy through the perineum to the prostate. One example of a therapy transducer 100 configured to deliver Histotripsy therapy to the prostate is shown in FIG. 1. The transducer 100 can comprise a plurality of ultrasound transducer elements 102 disposed within housing 104. The transducer can be connected to a waveform generator configured to deliver Histotripsy waveforms from the transducer to tissue. The prostate depth from this approach is significantly deeper than in the canine model above. Additionally, the skeletal anatomy of the pelvis and transrectal position of the ultrasound imaging probe significantly reduced the effective transducer aperture. A cut-out 106 in the lower perimeter of housing can be configured to accommodate an ultrasound imaging probe (not shown) which has an F-number=0.85 in the main diameter and F-number=0.98 at the cut out.

Based on bench-top experimentation and modeling, an initial set of therapy transducer excitation parameters (3 cycles/pulse, 750 Vpp, 500 Hz PRF (Pulse Repetition Frequency)) was selected for canine testing with this transducer. This excitation sequence produced a non-linear focal pressure waveform with a peak negative and peak positive pressure of approximately 25 MPa and 100 MPa in water. We define this sequence and its variants as a, standard, or non-optimized, sequence because the sequence parameters were not optimized for bubble cloud formation.

This standard excitation sequence and variants were used to treat approximately 30 canine subjects to establish feasibility, dosing (cumulative number of pulses), and treatment implementation guidelines. An additional 10 canine subjects were then treated in a confirmatory study. Although, these studies yielded outstanding efficacy results, the observation of apparent minor injury (subclinical fibrosis) to the prefocal abdominal rectus muscle in 2 of 10 subjects in the confirmatory trial led to the conclusion that the safety profile needed to be improved by developing Histotripsy pulse sequences that deliver energy more efficiently. It is likely that the need to improve the efficiency of Histotripsy will become more important as transducers are developed to go deeper into tissues through skeletal anatomical obstructions.

### SUMMARY OF THE DISCLOSURE

Improved efficiency leading to pre-focal heat reduction is imperative when soft tissue is targeted deep beneath the skin surface through skeletal anatomical obstructions which require ultrasound therapy transducers that have relatively high F numbers (F-number>0.8). Sequences optimized for enhanced Histotripsy homogenization of soft tissues were developed to reduce the potential of pre-focal thermal injury by optimizing the sequence efficiency. Improved efficiency of optimized excitation sequences increases the probability of initiating Histotripsy bubble clouds in tissue and reduces the occurrences of extinguishing bubble clouds when translating through tissues. Additionally, optimized sequences can be designed to selectively ablate fibrous tissues or ablate less dense tissues while preserving more fibro-elastic vital structures such as neuro-vascular structures.

Effective optimized sequences for high F-number transducers are characterized by an initiation pulse which is designed to create a least a single acoustically generated nucleus (bubble), followed by a shock scattering pulse (hereafter referred to as a scattering pulse or scattering pressure waveform) after an optimized time delay to enable a shockwave to impinge upon the first bubble to create a bubble cloud. Subsequent scattering pulses can follow also with optimized timing in order to further maintain the effectiveness of the bubble cloud. Note that pulse and pressure waveform will be used interchangeably in this application.

A method of treating tissue with ultrasound energy, comprising the steps of delivering an initiation pressure waveform from an ultrasound therapy transducer into tissue, the initiation pressure waveform being configured to produce at least one bubble in the tissue, delivering a scattering pressure waveform from the ultrasound therapy transducer into the at least one bubble within a life-cycle of the at least one bubble, and producing cavitation nuclei near the at least one bubble with the scattering pressure waveform.

In some embodiments, the scattering pressure waveform is delivered within 5 μs to 200 μs of the initiation pressure waveform.

In one embodiment, the method further comprises repeating the delivering an initiation pressure waveform and delivering a scattering pressure waveform steps until treatment of the tissue is completed.

In one embodiment, a pressure amplitude and/or number of cycles of the initiation pressure waveform is minimized to reduce tissue heating.

In another embodiment, a peak-to-peak pressure of the scattering pressure waveform is sufficient in amplitude create additional cavitation nuclei in the focal region.

In alternative embodiments, the pressure amplitude and/or number of cycles of the scattering pressure waveform is minimized to reduce tissue heating.

In some embodiments the method further comprises, after delivering the scattering pressure waveform, delivering a second scattering pressure waveform towards the at least one bubble and the cavitation nuclei.

In some embodiments, the second scattering pressure waveform is delivered within 5 μs to 1 s of the scattering pressure waveform.

In another embodiment, the method further comprises delivering additional scattering pressure waveforms without delivering additional initiation pressure waveforms until the at least one bubble and/or the cavitation nuclei no longer remain in the tissue.

In some embodiments, the additional scattering pressure waveforms are delivered every 5 μs to 1 s.

In one embodiment, a pulse sequence comprising the initiation pressure waveform and the scattering pressure waveform has a sequence PRF ranging from 1-5000 Hz.

In other embodiments, the scattering pressure waveform delivers less energy to intervening tissue than the initiation pressure waveform.

In one embodiment, the initiation pressure waveform and the scattering pressure waveform have substantially similar pressure amplitudes. In another embodiment, a pressure amplitude of the scattering pressure waveform is less than a pressure amplitude of the initiation pressure waveform. In alternative embodiments, a pressure amplitude of the scattering pressure waveform is more than a pressure amplitude of the initiation pressure waveform.

A method of treating tissue with ultrasound energy is provided, comprising the steps of transmitting an initiation pressure waveform from an ultrasound therapy transducer into tissue, the initiation pressure waveform being configured to produce at least one bubble in the tissue, during a life-cycle of the at least one bubble, transmitting a scattering pressure waveform from the ultrasound therapy transducer into the at least one bubble, the scattering pressure waveform configured to become a shocked focal pressure waveform in the tissue having a shocked positive pressure half cycle and a shocked negative pressure half cycle, the shocked positive pressure half cycle being configured to impinge on the at least one bubble and to scatter, invert, and constructively interfere with the shocked negative pressure half cycle to form a negative pressure half cycle waveform, and producing cavitation nuclei near the at least one bubble with a shock scattering mechanism between the positive pressure half cycle waveform and the at least one bubble.

A method of delivering ultrasound energy to tissue is provided, comprising the steps of delivering an initiation pulse from an ultrasound therapy transducer configured to provide at least 5 MPa of peak negative pressure to produce at least one bubble in the tissue, delivering a first scattering pulse into the at least one bubble within 5 μs to 200 μs of the initiation pulse, and producing a cavitation cloud of nuclei near the at least one bubble with a shock scattering mechanism between the first scattering pulse and the at least one bubble.

An ultrasound therapy system is provided, comprising an ultrasound therapy transducer, and an ultrasound therapy generator coupled to the ultrasound therapy transducer, the ultrasound therapy generator configured to drive the ultrasound therapy transducer to deliver an initiation pressure waveform into tissue to produce at least one bubble in tissue, the ultrasound therapy generator being further configured to drive the ultrasound therapy transducer to deliver a first scattering pressure waveform within 5 μs to 200 μs of the initiation pressure waveform into the at least one bubble to produce cavitation nuclei near the at least one bubble.

In some embodiments, a peak to peak pressure of the first scattering pulse is sufficient in pressure amplitude to produce cavitation nuclei near the at least one bubble.

In other embodiments, the ultrasound therapy generator is further configured to drive the ultrasound therapy transducer to deliver at least one additional scattering pulse after the first scattering pressure waveform to produce cavitation nuclei near the at least one bubble.

In one embodiment, the ultrasound therapy generator further comprises a controller configured to generate complex waveforms to initiate the initiation and scattering pressure waveforms, a high voltage power supply coupled to the controller, an amplifier configured to receive and amplify the complex waveforms from the controller and high voltage power supply, and a matching network configured to match an impedance of the ultrasound therapy transducer to the amplifier.

A method of treating tissue with ultrasound energy is provided, comprising the steps of producing at least one bubble in the tissue with ultrasound energy, colliding a shocked focal pressure waveform with the at least one bubble, and forming cavitation nuclei near the at least one bubble.

In one embodiment, the colliding step is performed during a life-cycle of the at least

one bubble.

In another embodiment, the colliding step is performed within 5 μs to 200 μs of the producing step.

In an alternative embodiment, the forming cavitation nuclei step is achieved with a shock scattering mechanism between the shocked focal pressure waveform and the at least one bubble.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is an ultrasound therapy transducer according to one embodiment.

FIGS. 2a-2c are illustrations of bubble cloud initiation in water.

FIG. 3. illustrates a focal pressure waveform according to one embodiment.

FIGS. 4a-4e are conceptual drawings that illustrate shock scattering.

FIGS. 5a-5c illustrate various embodiments of pulse sequences that include initiation and scattering pressure waveforms for delivering ultrasound energy to tissue.

FIG. 6 illustrates a system configured to deliver the preferred sequences for treating the tissue with cavitation.

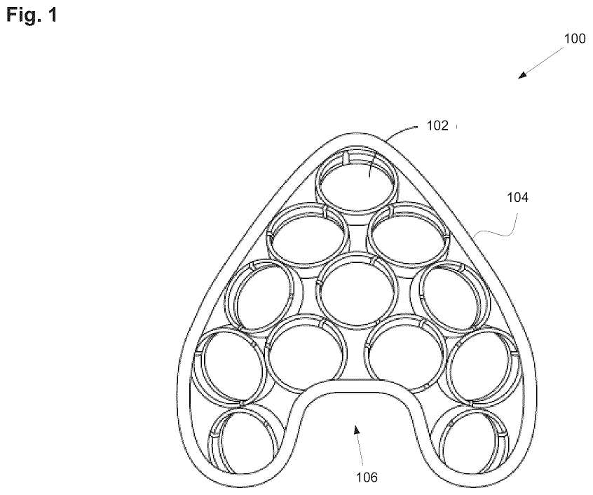

### DETAILED DESCRIPTION

### Generation of Cavitation

Several principles of cavitation nuclei and bubble cloud formation that provide important background information for the development of the preferred embodiment are disclosed herein. Cavitation nuclei are individual bubbles formed as a result of the delivery of low pressure to tissue. Bubble clouds can comprise of dense clusters of cavitation nuclei that form at or near the transducer focus. The formation of cavitation nuclei (bubble clouds) are both key components of Histotripsy therapy.

### Probability for Forming Cavitation Nuclei

Cavitation nuclei can be formed in tissue if the tissue is subjected to a peak negative (peak rarefaction) pressure approaching or exceeding the pressure level needed to create at least a single cavitation nucleus (bubble). Note that this level is variable and is dependent upon multiple factors including tissue properties (structure and composition, dissolved gas content, and existence of impurities), transducer geometry (focal distance and f number), and sequencing scheme (PRF; number of cycles). The number of cavitation nuclei formed from one acoustic pulse has been shown to be directly related to the peak negative pressure achieved.

### Cavitation Time Course

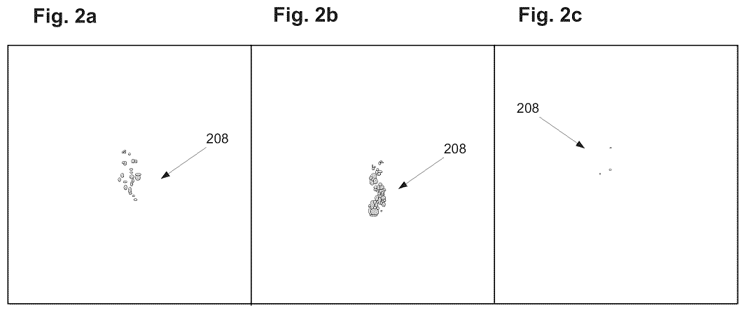

Cavitation nuclei grow to a maximum size and then collapse. The cavitation time course for the process of bubble initiation, growth, and then collapse is dependent on the medium (i.e., tissue type). The cavitation time course for liquids is longer than in gelatin and soft tissue. Table 1 compares cavitation initiation, growth, and collapse times in water vs. gelatin. FIGS. 2a-2c are illustrations showing a typical cavitation time course. FIG. 2a illustrates initiation of cavitation 208 in a medium, such as in tissue, in water, or in gelatin. FIG. 2b shows growth of the cavitation 208 to a maximum size, in which the cavitation bubbles are grouped together in the focal zone. FIG. 2c illustrates collapse of the cavitation 208 where nearly all the cavitation bubbles have collapsed and disappeared.

| Event/Delay-time | In Water (μs) | In Gelatin (μs) |
|---|---|---|
| Initiation | 68 | 68 |
| Growth | 149 | 84 |
| Collapse | 230 | 100 |

### Acoustic Shock and the Shock Scattering Mechanism for Bubble Cloud Formation

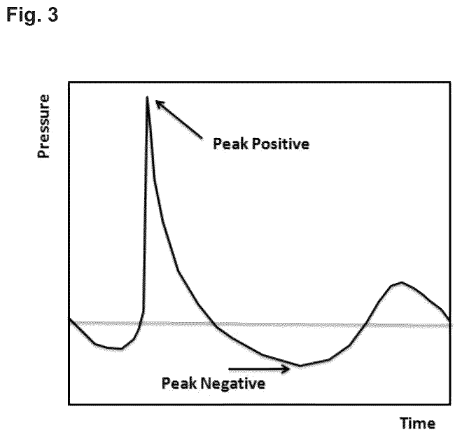

As a sound waveform travels through the medium the positive (compression) half cycle(s) travel faster than the negative (rarefaction) half cycle(s). This effect causes the pressure waveform to become nonlinear creating a sharp transition between negative and positive half cycles of the pressure waveform. The pressure amplitude of the positive half cycle increases as the slope of this transition increases and the pressure waveform is said to be become more nonlinear or “shocked”. This can be referred to as a shocked focal pressure waveform. The level of nonlinearity is dependent upon the pressure amplitude of the pressure waveform as well as the distance propagated through the medium. FIG. 3 shows an example of a shocked focal pressure waveform with a positive half cycle and a negative half cycle. It should be understood that shocked focal pressure waveforms can include a plurality of positive and negative half cycles.

According to the present disclosure, cavitation nuclei can be formed in tissue as a result of shock scattering. Shock scattering occurs when a shocked positive pressure half cycle of an acoustic waveform is reflected, or scattered, off of a pre-existing bubble(s) and the shocked positive pressure half cycle is consequently inverted such that it combines with the incident negative pressure half cycle of the acoustic waveform in an additive fashion. If this combined new negative pressure half cycle produced is large enough (i.e., above the intrinsic threshold for the tissue or medium of interest-greater than 5 MPa peak negative pressure for example), additional cavitation nuclei will form near any preexisting nuclei. This process repeats itself until the combined new negative pressure half cycle is not sufficient in pressure to create new cavitation nuclei.

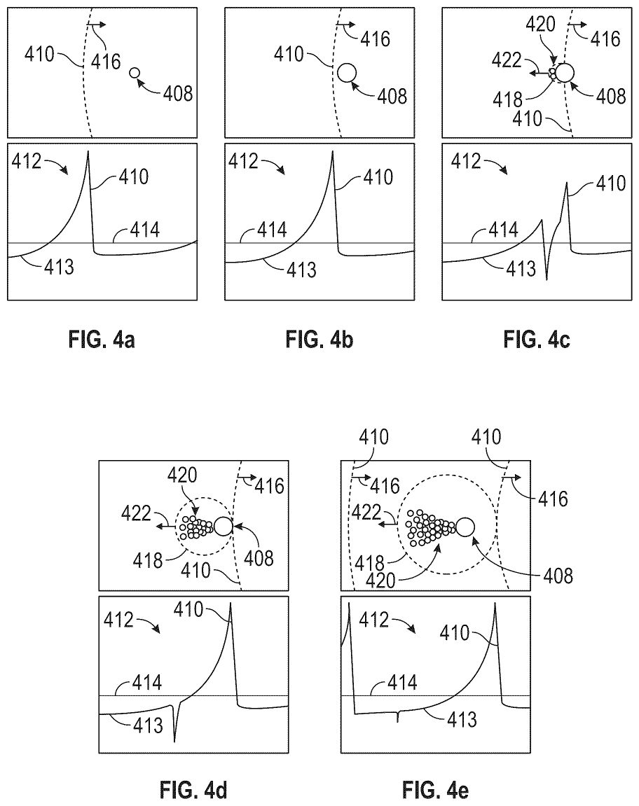

FIGS. 4a-4e are conceptual drawings illustrating a shock scattering method of Histotripsy therapy. The frames on the top show a pre-existing bubble 408 and a shocked positive pressure half cycle 410, and the frames on the bottom show the ultrasound pulse pressure distribution 412 (horizontal line 414 indicates a pressure amplitude of zero). The pre-existing bubble 408 may be formed with an initiation pulse or sequence as described above. A shocked pressure waveform can then be transmitted towards the bubble 408 during a life-cycle of the bubble according to one embodiment of the shock scattering method.

In FIGS. 4a-4e, the incident shocked pressure waveform 412 propagates from left to right towards the pre-existing bubble 408, as indicated by arrows 416. The incident shocked pressure waveform can be delivered towards and into the bubble during a life-cycle of the bubble, so that the incident shocked pressure waveform interacts with the bubble. A single pre-existing bubble 408 is shown in FIG. 4a, having already been generated in the tissue as described above. That bubble can expand in size, as shown in FIG. 4b, due to the initial negative pressure half cycle of the incident shocked pressure waveform. In FIG. 4c, a shocked positive pressure half cycle 410 of the incident shocked pressure waveform 412 impinges on the bubble 408 and the positive pressure half cycle begins to scatter. The scattered shocked positive pressure half cycle inverts and constructively interferes with the shocked negative pressure half cycle 413 of the incident shocked pressure waveform 412 to create a transient, large amplitude, negative pressure half cycle 418 (illustrated as the circular dotted line 418 in FIGS. 4c-4e) that produces additional cavitation nuclei 420 near or behind the bubble 408. The negative pressure half cycle 418 propagates from right to left, as indicated by arrows 422. The additional cavitation nuclei 420 form in the opposite direction of the shocked positive pressure waveform 410, until the negative pressure half cycle 418 drops below the threshold for the formation of cavitation nuclei, as shown in FIG. 4e. This process may be repeated with successive shocked pressure waveforms transmitted towards and into the pre-existing bubble 408 and additional cavitation nuclei 420.

Cavitation nuclei formed by this shock scattering method tend to grow towards the therapy transducer and their extent depends on the number of high pressure cycles in the pulse (waveform) and the pulse repetition frequency (PRF). Minimizing the number of cycles in a shocked waveform or reducing the sequence PRF are effective ways of reducing the length of the bubble cloud and also reducing the time average intensity and therefore the thermal dose.

### Enhanced Bubble Cloud Formation Using Shock Scattering

The key components of a preferred Histotripsy excitation sequence described in this disclosure are: 1) A first pulse of the sequence, referred to as an initiation pulse or initiation pressure waveform, configured to form at least one bubble in the tissue 2) A second pulse of the sequence, referred to as a scattering pulse or scattering pressure waveform, configured to generate cavitation nuclei near the at least one bubble through shock scattering, and 3) A specific time delay between the initiation and scattering pulses.

The key parameters for the pulses are: The initiation pulse should be configured to produce at least one bubble in the tissue of interest. This can be achieved with a traditional Histotripsy initiation pulse, as described above, or with other ultrasound techniques that can induce bubble formation in tissue due to boiling such as HIFU or boiling Histotripsy. The scattering pulse should have a peak-to-peak pressure high enough for shock scattering formation of cavitation nuclei. In some embodiments, the time delay between these pulses can range between 5 μs and 200 μs. In another embodiment, the time delay between these pulses can range between 5 μs and 40 ms. In another embodiment, the time delay between these pulses can range between 5 μs and 1 s.

In another embodiment, the pressure amplitude and/or number of cycles used in the initiation pulse can be increased or decreased. Increasing the pressure amplitude and/or number of cycles in the initiation pulse may increase the probability of creating cavitation in the tissue. However this would also likely increase the time averaged intensity, and thermal dose, delivered to the tissue and the extent of the bubble cloud. Decreasing the pressure amplitude and/or number of cycles of the initiation pulse will reduce the intensity, and thermal dose, of the sequence but may limit the ability of the sequence to generate and/or maintain cavitation.

In another embodiment, the pressure amplitude and/or number of cycles used in the scattering pulse(s) can be increased or decreased. Increasing the pressure amplitude and/or number of cycles in the scattering pulse(s) may increase the probability of creating cavitation in the tissue. However this would also likely increase the time averaged intensity delivered to the tissue, and thermal dose, delivered to the tissue and the extent of the bubble cloud. Decreasing the pressure amplitude and/or number of cycles of the scattering pulse(s) will reduce the intensity, and thermal dose, of the sequence but may limit the ability of the sequence to generate and/or maintain cavitation.

The sequence PRF can be as high as 5000 Hz assuming that the time averaged intensity, and resultant thermal dose, are kept within safe limits. The preferred range depends on the tissues being treated. A higher PRF is recommended for more dense and fibrous tissues, and a low PRF is recommended for less dense tissues and for preservation of more fibrous and often vital tissues. Selective treatment of tissues with Histotripsy based on their stiffness can be a probable design and performance consideration for sequence development.

In some embodiments additional scattering pulses with lower pressure amplitude and/or number of cycles (compared with the initiation pulse pressure amplitude and/or number of cycles), can be applied in order to reduce the intensity, and thermal dose, of the sequence without reducing the sequence PRF.

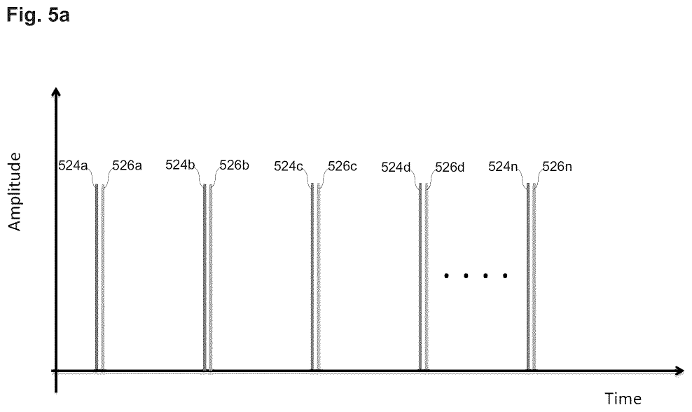

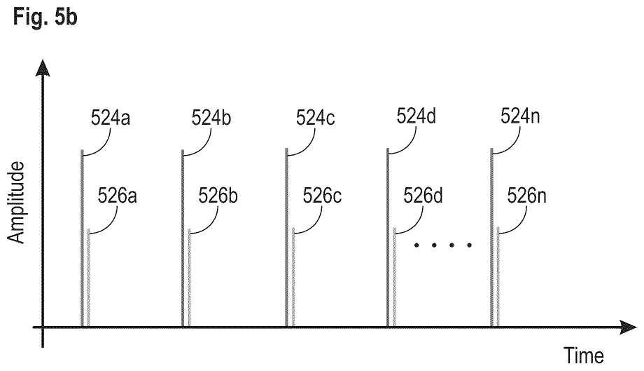

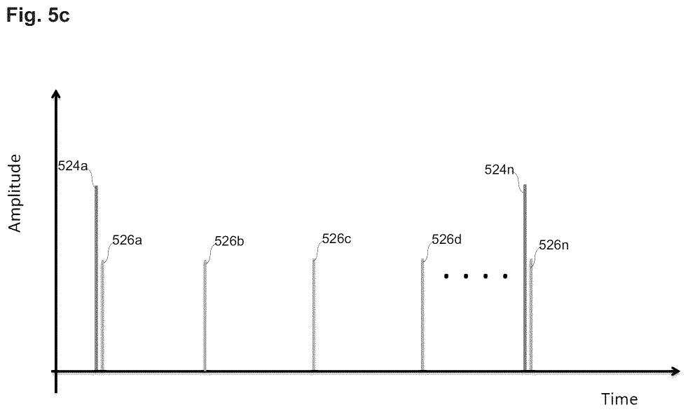

FIGS. 5a-5c illustrate three different embodiments for Histotripsy initiation and scattering pulse sequences that can be used to generate and maintain cavitation in tissue during a shocked scattering method of Histotripsy therapy. In FIG. 5a, an initiation pulse 524a comprising a pressure waveform configured to form at least one bubble in the tissue can be transmitted into tissue. After a specific time delay has passed, a scattering pulse 526a can be transmitted into tissue towards and into the at least one bubble formed by the initiation pulse 524a. In some embodiments, the specific time delay between these pulses can range between 5 μs and 200 μs. In another embodiment, the time delay between these pulses can range between 5 μs and 40 ms. In another embodiment, the time delay between these pulses can range between 5 μs and 1 s. The scattering pulse 526a becomes a shocked focal pressure waveform as it travels through the tissue, and the at least one shocked positive pressure half cycle of the scattering pulse impinges on the at least one bubble and is scattered by the at least one bubble. The shocked positive pressure half cycle of the scattering pulse inverts and constructively interferes with the shocked negative pressure half cycle of the scattering pulse to create a transient, large amplitude, negative pressure half cycle that produces additional cavitation nuclei behind the at least one bubble generated by the initiation pulse. These pulse sequence pairs of initiation and scattering pulses can be repeated to achieve the desired ablation effect in tissue from the resulting cavitation, as shown in FIG. 5a (pulse pairs 524b/526b, 524c/526c, 524d/526d, . . . , 524n/526n). In this embodiment, the pressure amplitudes and/or number of cycles of both the initiation and scattering pulses can be the same or approximately the same.

FIG. 5b shows another embodiment, similar to the embodiment of FIG. 5a, except the pressure amplitude of the scattering pulses 524a-524n are smaller than the pressure amplitude of the corresponding initiation pulses. Due to the principle of shock, the peak positive wave is amplified relative to the peak negative wave and therefore, the pressure amplitude used to create the scattering pulses can be lowered while still delivering the needed negative pressure with the reflected and inverted positive wave. This embodiment is more efficient than the embodiment of FIG. 5a and delivers a lower dose of energy into the tissue. In another embodiment, however, the pressure amplitude of the scattering pulses can be greater than the pressure amplitude of the corresponding initiation pulses.

FIG. 5c illustrates another embodiment, which is a variation of the embodiment of FIGS. 5a and 5b. In this embodiment, initiation pulse 524a is followed by a scattering pulse 526aafter a specific time delay, but instead of following that with another initiation/scattering pulse pair as in FIG. 5a, instead the scattering pulse 526a is followed with another scattering pulse 526bafter a second time delay. A plurality of scattering pulses can be delivered into tissue after the appropriate time delay to maintain the effectiveness of the bubble cloud (e.g., pulses 526c, 526d) to achieve the desired ablation effect in tissue from the resulting cavitation. The pressure amplitudes of the scattering pulse can be less than, equal to, or greater than the pressure amplitude of the initiation pulse. In some embodiments, the time delay for subsequent scattering pressure waveforms can be different than the time delay used for the first scattering pressure. For example, the first scattering pressure waveform may be delivered within 5 μs to 200 μs of the initiation pressure waveform, but subsequent scattering pressure waveforms may be delivered within 5us to 200 us, 5 μs to 40 ms, or 5 μs to 1 s. If the cavitation needs to be re-initiated in the tissue, the sequence can be re-started with another initiation/scattering pulse pair, as shown by 524n/526n in FIG. 5c. This embodiment also uses a lower pressure amplitude scattering pulse, as in the embodiment of FIG. 5b, but also uses fewer initiation pulses. The result of this embodiment is the lowest dose of energy delivered to tissue between the embodiments of FIGS. 5a-5c. This strategy has the potential to lower the dose significantly (as much as 50% for example) compared with traditional histotripsy sequences.

### Amplitude Reduction or Elimination of the Initiation Pulse Once the Bubble Cloud is Established:

The purpose of the initiation/scattering pair is to generate cavitation in tissue with shock scattering. Once the bubble cloud is generated, and if the focus is not moved, the initiation pulse may no longer be needed to maintain the effectiveness of the bubble cloud. In this case, the system could be designed to first create a bubble cloud with an initiation/scattering pair and follow that with lower pressure amplitude (relative to the initiation pulse pressure amplitude) scattering pulses until the focus is moved. At which point the process is repeated.

### System Software and Hardware Design that Allowed for Sequence Development

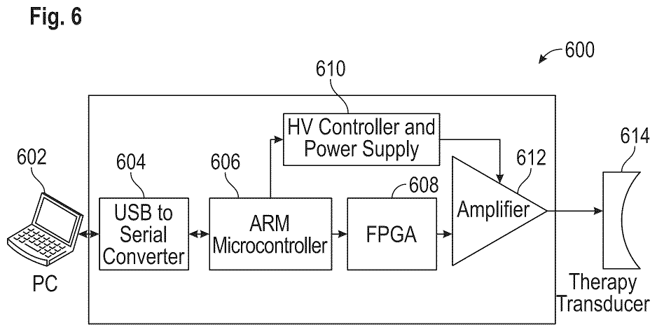

A Histotripsy system and generator is configured to generate very complex waveforms in order to support the ultrasound pulse sequences described herein. A simplified block diagram of system 600 is shown in FIG. 6. The main components of the system are: Computer/controller 602, USB to Serial Converter 604, Microcontroller 606, FPGA (Field Programmable Gate Array) 608, High Voltage Controller and Power Supply 610, Amplifier 612, and Therapy Transducer 614.

All controls for the generator can be established using “Histotripsy Service Tool” software that can run on the computer/controller 602 (e.g., a standard PC) and communicates to the generator via USB serial communication 604.

The system 600 is configured to receive multiple sets of different driving parameters and loop them, which give the ability to the user to create wide range of custom sequences where all parameters (PRF, voltage amplitude, number of cycles, number of pulses per set, frequency, transducer element channels enabled, and time delays) can be set differently for every pulse generated. Time delays between pulses can be specified by the PRF for a parameter set or by specifying zero as the number of cycles per pulse.

For overall voltage amplitude regulation, level of high voltage is changed accordingly through the Microcontroller 606 and HV Controller 610. This method cannot be used for dynamic voltage amplitude changes between two pulses since it will take too long for all capacitors on the HV line to discharge. For dynamic voltage amplitude changes between pulses, PWM (pulse width modulation) is used at the FPGA 608 where the duty cycle of the pulse is modulated in order to produce the desired pulse voltage and resultant pressure amplitude.

### Histotripsy Service Tool

Histotripsy Service Tool is an application that can be run on any PC and is used for controlling the system. The Histotripsy Service Tool can start/stop the therapy, set and read the level of high voltage, therapy parameters (PRF, number of cycles, duty ratio, channel enabled and delay, etc), and set and read other service and maintenance related items.

### USB to Serial Converter

USB to Serial converter 604 converts USB combination to serial in order to communicate to the Microcontroller 606.

### Microcontroller

The Microcontroller 606 communicates to the computer/controller 602 (Histotripsy Service Tool) to set/read working parameters, start/stop the therapy, etc. It can use internal flash memory to store all the parameters. The Microcontroller communicates to the FPGA 608 all driving parameters that are necessary to generate complex pulsing. It also communicates using serial communication to the high voltage controller and power supply 610 where it can set/read the proper level of driving voltage.

### FPGA

The FPGA 608 receives the information from the Microcontroller 606 and it generates the complex pulsing sequence that is required to drive the amplifier 612. The FPGA can run on 100 MHz clock since speed of pulsing is critical to be timed in 10 ns increments.

### High Voltage Controller and Power Supply

The High Voltage Controller and Power Supply 610 receives the commands from the Microcontroller 606 regarding the level of DC voltage that needs to be supplied to the amplifier circuitry in order to have an adequate voltage amplitude level at the output of the amplifier.

### Amplifier

The Amplifier 612 receives pulses generated by the FPGA and is supplied with high voltage from High Voltage Controller and Power Supply. It generates high voltage amplitude pulses that are fed to the Therapy Transducer 614 through the matching network components which properly matches the impedance of the therapy transducer to the impedance of the amplifier. It is necessary to use a large number of capacitors that can store enough energy to support peak current demand during the generation of high voltage amplitude pulses.

The data structures and code described in this detailed description are typically stored on a computer-readable storage medium, which may be any device or medium that can store code and/or data for use by a computer system. The computer-readable storage medium includes, but is not limited to, volatile memory, non-volatile memory, magnetic and optical storage devices such as disk drives, magnetic tape, CDs (compact discs), DVDs (digital versatile discs or digital video discs), or other media capable of storing computer-readable media now known or later developed.

The methods and processes described in the detailed description section can be embodied as code and/or data, which can be stored in a computer-readable storage medium as described above. When a computer system reads and executes the code and/or data stored on the computer-readable storage medium, the computer system performs the methods and processes embodied as data structures and code and stored within the computer-readable storage medium.

Furthermore, the methods and processes described above can be included in hardware modules. For example, the hardware modules can include, but are not limited to, application-specific integrated circuit (ASIC) chips, field-programmable gate arrays (FPGAs), and other programmable-logic devices now known or later developed. When the hardware modules are activated, the hardware modules perform the methods and processes included within the hardware modules.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An ultrasound therapy system, comprising:
an ultrasound therapy transducer; and
a waveform generator coupled to the ultrasound therapy transducer, the waveform generator being configured to drive the ultrasound therapy transducer to:
deliver a first pulse of a histotripsy excitation sequence into tissue, the first pulse comprising an initiation pressure waveform configured to produce at least one bubble in the tissue;
deliver a second pulse of the histotripsy excitation sequence into tissue, the second pulse comprising a scattering pressure waveform configured to interact with the at least one bubble within a life-cycle of the at least one bubble and further to produce a cavitation nuclei near the at least one bubble.

2. The system of claim 1, wherein the waveform generator is configured to drive the ultrasound therapy transducer to deliver the scattering pressure waveform within 5 μs to 200 μs of the initiation pressure waveform.

3. The system of claim 1, wherein the waveform generator is configured to drive the ultrasound therapy transducer to repeatedly deliver the initiation pressure waveform and the scattering pressure waveform until treatment of the tissue is completed.

4. The system of claim 1, wherein a peak-to-peak pressure of the scattering pressure waveform is sufficient in amplitude to create additional cavitation nuclei in the focal region.

5. The system of claim 1, wherein the waveform generator is configured to drive the ultrasound therapy transducer to deliver a second scattering pressure waveform towards the at least one bubble and the cavitation nuclei after delivering the scattering pressure waveform.

6. The system of claim 5, wherein the waveform generator is configured to drive the ultrasound therapy transducer to deliver the second scattering pressure waveform within 5 μs to 1 s of the scattering pressure waveform.

7. The system of claim 5, wherein the waveform generator is configured to drive the ultrasound therapy transducer to deliver additional scattering pressure waveforms without delivering additional initiation pressure waveforms until the at least one bubble and/or the cavitation nuclei no longer remain in the tissue.

8. The system of claim 7, wherein the waveform generator is configured to drive the ultrasound therapy transducer to deliver additional scattering pressure waveforms every 5 μs to 1 s.

9. The system of claim 1, wherein the histotripsy excitation sequence comprising the initiation pressure waveform and the scattering pressure waveform has a sequence pulse repetition frequency ranging from 1-5000 Hz.

10. The system of claim 1, wherein the scattering pressure waveform delivers less energy to intervening tissue than the initiation pressure waveform.

11. The system of claim 1, wherein the initiation pressure waveform and the scattering pressure waveform have similar pressure amplitudes.

12. The system of claim 1, wherein a pressure amplitude of the scattering pressure waveform is less than a pressure amplitude of the initiation pressure waveform.

13. The system of claim 1, wherein a pressure amplitude of the scattering pressure waveform is more than a pressure amplitude of the initiation pressure waveform.
